(19) United States
(12) Patent Application Publication
(10) Pub. No.: US 2024/0424055 A1
(43) Pub. Date: Dec. 26, 2024

(54) ADMINISTRATION OF C5-BINDING PROTEINS

(71) Applicant: IPC Research, LLC, New Haven, CT (US)

(72) Inventors: Eric Jacob Watsky, Guilford, CT (US); Zubin Minoo Bhagwagar, North Haven, CT (US); Haren Ashwanikumar Vasavada, Hamden, CT (US); Stephen Uden, East Lyme, CT (US)

(21) Appl. No.: 18/706,625

(22) PCT Filed: Nov. 1, 2022
(86) PCT No.: PCT/US2022/079010

§ 371 Date: May 1, 2024

Related U.S. Application Data
(60) Provisional application No. 63/274,480, filed on Nov. 1, 2021.

(51) Int. Cl. A61K 38/17

(52) U.S. Cl. CPC A61K 38/1709

(57) ABSTRACT

Methods are described for administering a C5-binding protein to a subject. The methods include subcutaneously administering a pharmaceutical composition comprising a C5-binding protein to a subject in certain amounts and/or using certain dosing schedules to reduce the amount of free C5 in the subject and/or inhibit terminal complement activity in the subject.

### CROSS-REFERENCE TO RELATED APPLICATIONS

This application claims the benefit of priority of U.S. Provisional Patent Application No. 63/274,480 filed on Nov. 1, 2021, the content of which is hereby incorporated by reference in its entirety.

### SEQUENCE LISTING

The instant application contains a Sequence Listing which has been submitted electronically in XML file format and is hereby incorporated by reference in its entirety. Said XML file, created on Oct. 31, 2022, is named ST26-IPC.003.WO1-Oct 31.xml and is 1983 bytes in size.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products or active agents cited or mentioned herein are incorporated by reference. Documents incorporated by reference into this text, or any teachings therein, can be used in the practice of the present invention.

### BACKGROUND

The complement system is an instrumental part of the innate immune system that provides an immediate line of defense against microorganisms without pre-exposure. The complement cascade is activated by exogenous surfaces and danger- and pathogen-associated molecular patterns as well as antibodies/immune complexes through 3 separate routes, the classical, the lectin, and the alternative pathways, which converge at the level of complement component C3. Cleavage of C3 into C3a and C3b leads to the formation of a convertase that in turn cleaves complement component C5 into C5a and C5b. The anaphylatoxins C5a and C3a drive inflammation while C5b assembles with complement components C6, C7, C8, and C9 (C5b-9) to form the membrane attack complex (MAC) that lyses cells by forming a pore through the cell wall/membrane. MAC formation is particularly important in the defense against encapsulated bacteria, e.g., Neisseria meningitidis. In addition, upon activation, complement fragments (mainly from C3 and C4) opsonize surfaces for subsequent recognition by complement receptors on phagocytic cells and erythrocytes, thereby enabling the efficient clearance of opsonized microorganisms, immune complexes, and debris (Chen et al., “The complement system in systemic autoimmune disease,” J. Autoimmun. 2010; 34 (3): J276-J286; Walport, “Complement. First of two parts.” N. Engl. J. Med. 2001; 344(14):1058 1066; Walport, “Complement. Second of two parts.” N. Engl. J. Med. 2001; 344(15):1140 1144). The complement system is involved in the pathology of several disorders in different disease areas. Diseases with significant complement involvement include autoimmune diseases. Furthermore, mutations in complement proteins or dysfunctional regulation of complement are causative in several rare (or ultra-rare) conditions, in which hemolysis is a component in the pathology. Complement component C5 is common to all pathways of complement activation and blocking C5 inhibits the progression of the terminal complement cascade (downstream of C5) regardless of the stimulus. Inhibition of C5 thereby has the potential to prevent the deleterious properties of terminal complement activation while leaving the essential functions of the proximal complement cascade intact, i.e., opsonization of microorganisms and clearance of immune complexes.

Significant efforts have been made to modulate the activity of the complement pathway to treat complement-mediated diseases. For example, several antibodies against C5 have been developed for the treatment of a variety of complement-related diseases. However, such antibody-based therapeutics require administration of high doses and must be delivered by intravenous (IV) infusion (see, e.g., Lee et al., “Ravulizumab (ALXN1210) vs eculizumab in adult patients with PNH naive to complement inhibitors: the 301 study;” Blood. 2019 Feb. 7; 133(6): 530-539). There are also anti-C5 peptide therapeutics in clinical development. However, when administered subcutaneously these peptide therapeutics require daily administration (see, e.g., Sadik et al., “Evaluation of Nomacopan for Treatment of Bullous Pemphigoid A Phase 2a Nonrandomized Controlled Trial;” JAMA Dermatol. 2022; 158(6):641-649; and Howard et al., “Clinical Effects of the Self-administered Subcutaneous Complement Inhibitor Zilucoplan in Patients with Moderate to Severe Generalized Myasthenia Gravis: Results of a Phase 2 Randomized, Double-Blind, Placebo-Controlled, Multicenter Clinical Trial.” JAMA Neurol. 2020; 77(5):582-592).

Accordingly, there is a need in the art for new and improved C5 inhibitor therapies, including those that require lower doses and can be delivered by more convenient routes, such as by small volume subcutaneous injections, and those that do not require daily administration. The present invention addresses this need.

### SUMMARY OF THE INVENTION

Some of the main aspects of the present invention are summarized below. Additional aspects are described in the below Detailed Description of the Invention, Examples, and Claims sections of this patent disclosure. The description in each section of this patent disclosure is intended to be read in conjunction with the other sections. Furthermore, the various embodiments described in each section of this disclosure can be combined in various ways, regardless of any headings or subheadings, and all such combinations are intended to fall within the scope of the present invention.

The present invention involves a C5-binding protein from a class of engineered proteins, commonly referred to as affibodies, that are derived from the B domain in the immunoglobulin binding region of staphylococcal protein A (see Lofblom J, Feldwisch J, Tolmachev V, et al. “Affibody molecules: engineered proteins for therapeutic, diagnostic and biotechnological applications;” FEBS Lett. 2010; 584 (12):2670-2680). Like antibodies, these proteins can be engineered and/or selected to have affinity for a given protein of interest, for example using phage display-based library screens. The present invention involves such an engineered affibody protein that has C5-binding activity. In particular, the present invention provides various methods for administering this C5-binding protein to human subjects. Importantly, and as described further in the Examples section of this patent disclosure, clinical trial data obtained to-date shows that, when administered subcutaneously to human subjects, a composition containing this C5-binding protein leads to a clinically meaningful reduction in free C5 levels in the serum without the occurrence of any serious adverse events. This is particularly significant given that a prior clinical trial, in which a product containing a different C5-binding affibody molecule was administered to patients, was terminated as the result of adverse events without any evidence of target pharmacological activity or efficacy being reported (see, U.S. clinical trial identifier no. NCT02083666, see also Berglund & Stromberg; 2016; “The clinical potential of Affibody-based inhibitors of C5 for therapeutic complement disruption;” Expert Review of Proteomics, 13:3, 241-243). Furthermore, the clinical trial data obtained to-date shows that the C5-binding protein described herein exhibits desirable pharmacokinetic and pharmacodynamic properties at doses that are far lower than those required for anti-C5 antibody therapeutics and that enable clinically effective doses to be administered in low volume subcutaneous injections. In addition, the results presented herein show that serum concentrations of the C5-binding protein are relatively stable for at least 3 days following administration, having a serum terminal half-life of greater than 300 hours. The combination of these clinical features provides numerous advantages of the present C5-binding protein over both anti-C5 antibody therapeutics that require intravenous administration and anti-C5 peptide therapeutics that require daily administration.

Accordingly, in some embodiments the present invention provides methods of administering a C5-binding protein to a subject, such methods comprising administering to the subject a pharmaceutical composition comprising a C5-binding protein having the amino acid sequence of SEQ ID NO. 1, wherein the pharmaceutical composition is administered to the subject subcutaneously.

In some embodiments the methods of the present invention involve administration of a single dose of the pharmaceutical composition to the subject. In other embodiments the methods of the present invention involve administration of a series of multiple (two or more) doses of the pharmaceutical composition to the subject.

In some of those embodiments where a series of two or more doses of the pharmaceutical composition is administered to the subject, the doses are administered to the subject at approximately weekly intervals (QW).

In some of those embodiments where a series of two or more doses of the pharmaceutical composition is administered to the subject, the methods comprise administering one or more induction doses of the pharmaceutical composition and one or more maintenance doses of the pharmaceutical composition. In some embodiments the one or more maintenance doses are administered at weekly intervals (QW). In some embodiments four or more maintenance doses are administered to the subject—each at a weekly interval (QW). In some embodiments one induction dose of the pharmaceutical composition is administered—with the day on which the induction dose is administered being referred to herein as Day 1. In some embodiments two induction doses of the pharmaceutical composition are administered, for example with the first induction dose being administered on Day 1 and the second induction dose being administered on Day 4. In some embodiments three induction doses of the pharmaceutical composition are administered, for example with the first induction dose being administered on Day 1, the second induction dose being administered on Day 3, and the third induction dose being administered on Day 5.

In some embodiments the dose administered to the subject (whether that dose is the sole dose administered, a single dose within a series or multiple doses, an induction dose, or a maintenance dose) consists of from 2 mg to about 500 mg of the C5-binding protein. In some embodiments the dose administered consists of from about 50 mg to about 450 mg of the C5-binding protein. In some embodiments the dose administered consists of from about 100 mg to about 450 mg of the C5-binding protein. In some embodiments the dose of the C5-binding protein administered is about 30 mg. In some embodiments the dose of the C5-binding protein administered is about 40 mg. In some embodiments the dose of the C5-binding protein administered is about 50 mg. In some embodiments the dose of the C5-binding protein administered is about 60 mg. In some embodiments the dose of the C5-binding protein administered is about 70 mg. In some embodiments the dose of the C5-binding protein administered is about 75 mg. In some embodiments the dose of the C5-binding protein administered is about 80 mg. In some embodiments the dose of the C5-binding protein administered is about 90 mg. In some embodiments the dose of the C5-binding protein administered is about 100 mg. In some embodiments the dose of the C5-binding protein administered is about 125 mg. In some embodiments the dose of the C5-binding protein administered is about 150 mg. In some embodiments the dose of the C5-binding protein administered is about 200 mg. In some embodiments the dose of the C5-binding protein administered is about 250 mg. In some embodiments the dose of the C5-binding protein administered is about 300 mg.

In some embodiments, the C5-binding protein is administered to the subject at an amount and/or frequency effective to reduce the amount of free C5 in the subject. In some such embodiments, the C5-binding protein is administered to the subject at an amount and/or frequency effective to reduce the serum free C5 concentration in the subject to about 0.5 micrograms/mL or less. In some embodiments, the C5-binding protein is administered to the subject at an amount and/or frequency effective to reduce the serum free C5 concentration in the subject by about 99% or more as compared to the baseline serum free C5 concentration in the subject prior to administration of the C5-binding protein.

In some embodiments, the C5-binding protein is administered to the subject at an amount and/or frequency effective to inhibit activation of the terminal complement pathway in the subject.

In some embodiments, the C5-binding protein is administered to the subject at an amount and/or frequency effective to achieve a C_{trough} of the C5-binding protein of about 0.5 micromolar or more, or about 1.0 micromolar or more, or about 1.5 micromolar or more, or about 2.0 micromolar or more.

In some embodiments the C5-binding protein is administered to the subject at any of the amounts and/or frequencies described in the Examples section of this patent disclosure.

In some embodiments the present invention provides pharmaceutical compositions comprising a C5-binding protein having the amino acid sequence of SEQ ID NO. 1. In some embodiments the pharmaceutical compositions comprise a C5-binding protein having the amino acid sequence of SEQ ID NO. 1 and at least one additional pharmaceutically-acceptable component. In some embodiments the pharmaceutical compositions comprise one or more additional components selected from the group consisting of: histidine, arginine, polysorbate 20, and water. In some embodiments the pharmaceutical compositions comprise each of histidine, arginine, polysorbate 20, and water. In some embodiments the pharmaceutical compositions comprise about 20 mM histidine, about 150 mM arginine, about 0.05% polysorbate 20, and water. In some embodiments the pharmaceutical compositions comprise about 100 mg/ml of the C5-binding protein. In some embodiments the pharmaceutical compositions have a pH of about 7.0. In some embodiments the pharmaceutical compositions are stable when stored at from about 2° C. to about 8° C. In some embodiments the pharmaceutical compositions are stable when stored at from about 2° C. to about 8° C. for up to 18 months, or more. In some embodiments the pharmaceutical compositions are stable when stored at from about 15° C. to about 25° C. In some embodiments the pharmaceutical compositions are stable when stored at from about 15° C. to about 25° C. for up to about 36 hours, or more.

In some embodiments, the present invention provides an autoinjector device comprising a pharmaceutical composition as described herein. In some embodiments, the present invention provides an autoinjector device comprising a volume of a pharmaceutical composition as described herein sufficient for administration of a single dose of the C5-binding protein to a subject. In some embodiments, the present invention provides an autoinjector device comprising a volume of a pharmaceutical composition as described herein sufficient for administration of multiple doses of the C5-binding protein to a subject. In some embodiments, the present invention provides an autoinjector device comprising a pharmaceutical composition comprising the C5-binding protein at a concentration of about 100 mg/mL.

Further aspects, features, and advantages of the present invention will be better appreciated upon a reading of the following Detailed Description, Examples, and Claims sections of this patent disclosure.

### BRIEF DESCRIPTION OF THE DRAWINGS

FIG. 1. Graph showing ex vivo hemolytic inhibition data for the C5-binding protein described herein. The C5-binding protein (at 0.0625, 0.125, 0.25, 0.5, 1, and 2 μM) was incubated ex vivo with human serum and the % of hemolysis was determined using a standard assay. Complete hemolytic inhibition was achieved at concentrations of the C5-binding protein of 0.5 μM (and above). Percentage hemolytic activity is shown on the vertical axis and concentration of the C5-binding protein is shown on the horizontal axis.

FIG. 2. Graph showing pharmacokinetics (PK) data for healthy participant Cohorts 1-4 from the clinical trial described in Examples 2 and 3. The vertical axis shows the concentration of the C5-binding protein in the serum in micromolar (μM) units and the horizontal axis shows time post administration/treatment (“TPT”) in hours.

FIG. 3. Graph showing pharmacokinetics (PK) data for healthy participant Cohort 4 from the clinical trial described in Examples 2 and 3. The vertical axis shows the concentration of the C5-binding protein in the serum in micromolar (μM) units and the horizontal axis shows time post administration/treatment (“TPT”) in hours.

FIG. 4 A-B. Pharmacodynamic (PD) data from healthy participants in Cohorts 2-4 from the clinical trial described in Examples 2 and 3. FIG. 4 A and FIG. 4B show graphs in which the measured concentration of free C5 in the serum in ng/mL on the vertical axis is plotted against the measured concentration of the C5-binding protein in the serum in micromolar (μM) units on the horizontal axis. FIG. 4A contains data from 18 subjects. Each plotted data point in FIG. 4A (represented by an open circle) is averaged (mean) data from 6 subjects and each data point represents mean data obtained following administration of a specific administered dose of the C5-binding protein (either 10 mg, 30 mg, or 100 mg) and at a specific time post administration (either 0, 2, 4, 12, 24, or 72 hours post administration). In FIG. 4B each plotted data point (represented by an open circle) is averaged (mean) data from 6 subjects obtained following administration of a 100 mg dose of the C5-binding protein at a specific time post administration (either 0, 2, 4, 12, 24, or 72 hours post administration). These data show that, as the concentration of the C5-binding protein in the serum increases, the serum free C5 concentration decreases.

FIG. 5 A-E Graphs of concentration of C5-binding protein in the serum in micromolar (μM) units (on the vertical axis) plotted against time in hours (on the horizontal axis) for multiple administrations of 100 mg (FIGS. 5A-D) or 150 mg (FIG. 5E) of the C5-binding protein. The graphs show modeling data obtained using single dose clinical trial data as an input. The administration schedule modelled is indicated above each graph. The horizontal line plotted on each of the graphs indicates a 2 μM concentration of the C5-binding protein in the serum. In each graph the upper, dotted line represents the 97.5^{th} percentile of the C5-binding protein concentration, the middle, dotted line represents the 50^{th} percentile of the C5-binding protein concentration, and the lower, dotted line represents the 2.5^{th} percentile of the C5-binding protein concentration.

### DETAILED DESCRIPTION OF THE INVENTION

The present invention relates to a C5-binding protein, pharmaceutical compositions comprising the C5-binding protein, and various methods involving the administration of the C5-binding protein (or pharmaceutical compositions comprising the C5-binding protein) to subjects, as further described in the below Detailed Description section of this patent disclosure, as well as in the Summary of the Invention, Examples and Claims sections of this patent disclosure. The various embodiments described in this Detailed Description can be combined in various ways, regardless of any headings or subheadings herein.

The practice of the present invention will employ, unless otherwise indicated, conventional techniques of pharmaceutics, formulation science, immunology, hematology, cell biology, molecular biology, clinical pharmacology, and clinical practice, which are within the skill of the art.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Identification of references or other documents herein, or incorporation-by-reference of any thereof, is not an admission as to the prior art status of any of such references or documents.

In order that the present invention can be more readily understood, certain terms are defined below. Additional definitions are set forth throughout the disclosure. Unless defined otherwise herein, all technical and scientific terms used herein have the same meaning as commonly understood by one of ordinary skill in the art to which this invention is related.

### Definitions

The phraseology or terminology in this disclosure is for the purpose of description and not of limitation, such that the terminology or phraseology of the present specification is to be interpreted by the skilled artisan in light of the teachings and guidance provided herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising” or “having,” otherwise analogous embodiments described in terms of “consisting of” and/or “consisting essentially of” are included.

Units, prefixes, and symbols are denoted in their Systeme International de Unites (SI) accepted form.

Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

Where a numeric term is preceded by the qualifier “about,” the term includes the stated number and values ±10% of the stated number. For example, a concentration of about 1 mg/mL includes 0.9 mg/mL to 1.1 mg/mL. Furthermore, whenever a numeric term is preceded by the qualifier “about,” embodiments having the precise stated numeric value without the “about” qualifier are also contemplated and fall within the scope of the present invention. Conversely, whenever an embodiment of the present invention refers to a specific numeric term, otherwise analogous embodiments with an “about” qualification are also contemplated and fall within the scope of the present invention. Unless otherwise indicated, the term “about” preceding a series or list of elements is to be understood to refer to every element in the series or list. Similarly, unless otherwise indicated, the terms “at least” and “up to” preceding a series or list of elements is to be understood to refer to every element in the series.

The safety of drugs (which, as used herein, includes biologic agents) and methods of administration of drugs, is determined based on an assessment of parameters such as adverse events, vital signs, clinical laboratory values, and electrocardiogram (ECG) findings. The terms “adverse event” and “serious adverse event” are used herein consistently with their accepted meanings in the art and consistently with their use by the U.S. Food and Drug Administration. Accordingly, as used herein the term “adverse event” (“AE”) refers to any unfavorable and unintended sign (e.g., an abnormal laboratory finding) or symptom temporally associated with the use of a drug, whether or not considered related to the drug. A “mild” adverse event is an AE in which there is an awareness of symptoms that are easily tolerated, causing minimal discomfort and not interfering with everyday activities. A “moderate” adverse event is an AE in which there is sufficient discomfort to interfere with everyday activities. A “severe” adverse event is more medically significant than a mild or moderate adverse event but does not rise to the level of a “serious” adverse event. A “serious adverse event” (“SAE”) refers to an event that is fatal or immediately life-threatening; that requires inpatient hospitalization or prolongation of existing hospitalization; that results in persistent disability/incapacity; or that is a congenital anomaly/birth defect; or another medically important serious event that may jeopardize the subject or require medical or surgical intervention to prevent one of the other outcomes listed in this definition.

The terms “affinity” and “binding affinity” generally refer to the strength of the sum total of non-covalent interactions between a single binding site of a molecule and its binding partner (e.g., between the active agent of the present invention and the human complement protein C5). The affinity of a molecule X for its partner Y can generally be represented by the dissociation constant (KD). Affinity can be measured by common methods known in the art, e.g., flow cytometry, enzyme-linked immunosorbent assay (ELISA), radioimmunoassay (RIA), or kinetic analysis (e.g., KINEXA® or BIACORE™ or OCTET® analysis). Direct binding assays as well as competitive binding assay formats can be readily employed. The measured affinity of a particular binding pair interaction can vary if measured under different conditions (e.g., salt concentration, pH, temperature). Thus, measurements of affinity and other binding parameters (e.g., KD or Kd, Kon, Koff) are typically made with standardized solutions of binding partners and a standardized buffer, as known in the art.

“Binding” generally refers to the non-covalent interaction between a single binding site of a molecule and its binding partner (e.g., between the active agent of the present invention—which is a C5-binding protein—and the human complement protein C5).

The terms “C5,” C5 protein,” “complement C5” and “complement protein C5” and “complement C5 protein” are used interchangeably herein. The C5 protein, and its structure and biological function are well known in the art. The C5 protein is a component of the complement system and is common to all pathways of complement activation. Blocking C5 (e.g., using a C5 inhibitor) inhibits terminal complement activation while leaving the essential functions of the proximal complement cascade (such as opsonization of microorganisms and clearance of immune complexes) intact. For example, C5 inhibitors can inhibit the cleavage of C5 to C5a and C5b, preventing the generation of the potent pro-inflammatory anaphylatoxin C5a and inhibiting the assembly of nascent C5b, C6, C7, C8, and C9 molecules into the membrane attack complex.

An “effective amount” (for example of an active agent or a pharmaceutical composition) is an amount sufficient to effect a specifically stated purpose or biological or medicinal outcome or response in a subject.

The terms “induction dose,” “induction phase,” “maintenance dose,” and “maintenance phase” are standard terms in pharmacokinetics and are used herein consistently with their accepted meanings in the art. Typically, induction doses are doses given during the initial phase (induction phase) of a multiple dose course of treatment and constitute either administration of higher doses of the active agent than are administered during the subsequent maintenance phase or more frequent administration of the active agent than during the subsequent maintenance phase. Typically, induction doses are used to increase the speed with which an effective concentration of an active agent is achieved, for example, in the case of the present invention, to increase the speed with which a concentration of the C5-binding protein that is effective to reduce serum free C5 levels to a clinically meaningful degree is achieved. Typically, during the maintenance phase the concentration of an active agent (e.g., in the serum) remains relatively consistent over time (i.e., is maintained) with some cyclical variation between peak (Cₘₐₓ) and trough (C_{trough}) concentrations.

The terms “inhibit,” “block,” “reduce” and “suppress” are used interchangeably and refer to any statistically significant decrease in the stated occurrence or activity, including full blocking of the occurrence or activity. For example, “inhibition” can refer to a decrease of about 10%, 20%, 30%, 40%, 50%, 60%, 70%, 80%, 90%, 95%, 98%, 99% or 100% in the stated activity or occurrence. A determination of whether there is a statistically significant decrease in a stated occurrence or activity, or a determination of the degree of any decrease in a stated occurrence or activity, may be assessed in relation to any suitable comparator or control, for example in relation to the situation before the decrease occurred and/or in the absence of an agent that caused the decrease to occur.

The term “inhibitor” refers to an active agent that inhibits a stated occurrence or activity. The active agent of the present invention binds to C5 (and can thus be referred to as a C5-binding protein) and inhibits activation of the terminal complement cascade downstream of C5 (and can thus referred to as a complement inhibitor or C5 inhibitor).

The term “pharmaceutical composition” refers to a preparation that is in such form as to permit the biological activity of the active agent therein to be effective and which contains no additional components that are unacceptably toxic to a subject to which the composition would be administered. Such a composition can be sterile and can comprise a pharmaceutically acceptable carrier, such as water (e.g., water for injection) or a physiological saline. Suitable pharmaceutical compositions can comprise one or more of a buffer (e.g., acetate, phosphate, or citrate buffer), a surfactant (e.g., polysorbate), a stabilizing agent (e.g., polyol or amino acid), a preservative (e.g., sodium benzoate), and/or other conventional solubilizing or dispersing agents.

“Pharmacodynamics” or “PD” refers to the study of the molecular, biochemical, physiologic, and other biological effects of active agents in the bodies of subjects. In the case of C5 inhibitors, pharmacodynamics can be evaluated using metrics relating to the effects of administered agents on, for example, amount of bound C5 (i.e., C5 bound by the active agent), amount of free C5 (i.e., C5 not bound by the active agent), amount of total C5 (bound C5 plus free C5), proportion of total C5 that is free C5, cleavage of C5 by C5 convertase, production of C5a, production of C5b, production of other complement components downstream of C5 in the complement pathway, terminal complement pathway activity, etc. Where specific numerical values for PD parameters are provided herein, and unless stated otherwise, the values provided are for serum (typically as measured in a serum sample prepared from a blood sample obtained from a subject). However, it may be possible to obtain measurements of such PD parameters from plasma, blood, or blood-derived samples from a subject. Thus, where specific numerical serum PD values are provided, equivalent or comparable numerical PD values such as might be obtained from plasma, blood, or other blood-derived samples, also fall within the scope of the description. Several of the embodiments of the present invention involve the PD parameter of serum free C5. Methods of measuring serum free C5 are known in the art. In some embodiments specific serum free C5 concentrations are specified, e.g., a serum free C5 concentrations of 0.5 micrograms/mL or less. In some embodiments, instead of referring to a specific concentration of free C5 in the serum, the amount of free C5 in the serum is referred to in relative terms, typically as a % of a baseline concentration of free C5 that was present in the serum prior to administration of the C5-binding protein, e.g., an amount free C5 in the serum that represents a reduction by 99% or more as compared to the baseline concentration of free C5 in the serum prior to administration of the C5-binding protein. In some embodiments the amount of free C5 in the serum (whether described as a concentration or as a % of a baseline concentration) is the amount at a certain time after administration of a C5-binding protein, such as, for example, at 12, or 24, or 36, or 48 or 72 hours after administration of the C5-binding protein. In embodiments where a series of multiple doses of the C5-binding protein is administered to a subject, the amount of free C5 in the serum (whether described as a concentration or as a % of a baseline concentration) typically refers to the amount achieved during the maintenance phase of the administration protocol. In preferred embodiments the specified amount of free C5 in the serum (whether described as a concentration or as a % of a baseline concentration) refers to a threshold amount (e.g., 0.5 micrograms/mL or less, or a reduction by 99% or more) that is achieved throughout the maintenance phase, despite some variation in the concentration of the C5-binding protein in the serum between Cₘₐₓ and C_{trough}.

“Pharmacokinetics” or “PK” refers to the study of how an administered active agent is processed by the body of a subject. PK determinations include how the agent enters the blood circulation (absorption), is dispersed or disseminated throughout the fluids and tissues of the body (distribution), is recognized and transformed by the body (metabolism), and/or is removed from the body (excretion). Pharmacokinetics can be evaluated using various well-known metrics, many of which are calculated based on the quantity of the active agent in the body (or in a biological sample obtained or processed from the body) at various time points following the administration of the active agent. For example, “AUC” or “area-under-the-curve” (or area under the concentration-time curve) is a pharmacokinetics metric that describes the variation of the concentration of an active agent, typically in serum or plasma, as a function of time. AUC may be calculated for different periods of time, for example, from time zero to specified time t (AUCₜ or AUC₀₋ₜ), from time zero to infinity (AUC∞ or AUC0-∞), etc. “Cₘₐₓ” is the peak concentration of an active agent, typically in serum or plasma, after administration. “C_{trough}” is the lowest concentration of an active agent, typically in serum or plasma, after administration (typically the concentration at the end of a dosing interval—i.e., the concentration after a given dose in a series of doses is administered and just prior to administration the next dose in the series). Time after administration may be measured from T₀, which is the time that the active agent is administered. “Tₘₐₓ” refers to the time of after administration (which occurs at T₀) for the active agent to reach its maximum concentration (Cₘₐₓ). “T_{1/2}” refers to the half-life of the active agent, i.e., the time required for the concentration of the active agent to reach half of its original value. “CL” is the systemic clearance of the agent. V_{d} is the volume of distribution of the agent. Where specific numerical values for PK parameters are provided herein, and unless stated otherwise, the values provided are for serum (typically as measured in a serum sample prepared from a blood sample obtained from a subject). However, it may be possible to obtain measurements of such PK parameters from plasma, blood, or blood-derived samples from a subject. Thus, where specific numerical serum PK values are provided, equivalent or comparable numerical PK values such as might be obtained from plasma, blood, or other blood-derived samples, also fall within the scope of the description.

The terms “polypeptide,” “peptide,” and “protein” are used interchangeably to refer to polymers of amino acids, and their salts. Either the standard three-letter or one-letter amino acid abbreviations used in the art may be used herein to represent amino acid residues. Strings of amino acid abbreviations are used to represent peptides by their amino acid sequence. Unless indicated otherwise, proteins are indicated with the N-terminus on the left and the sequence is written from the N-terminus to the C-terminus. The terms “polypeptide,” “peptide,” and “protein”—as used herein—include monomeric and multimeric (e.g., dimeric) forms of the polymers of amino acids.

A “subject” or “patient” is an individual, particularly a mammalian individual, for whom prophylaxis or treatment (e.g., using an active agent, pharmaceutical composition, or method as described herein) is desired, needed or performed. A subject “in need thereof” is a subject who could reasonably be expected to benefit from prophylaxis or treatment using an active agent, pharmaceutical composition, or method as described herein—for example as determined by a medical professional. In some embodiments of the present invention the subject is any mammalian subject, including humans, domestic animals, farm animals, sports animals, and laboratory animals including, e.g., humans, non-human primates, canines, felines, porcines, bovines, equines, rodents, including rats and mice, rabbits, etc. In preferred embodiments the subject is a human.

### Active Agent

The present invention involves an active agent that is a C5-binding protein. It is a recombinant protein that comprises an affibody Z-domain and an albumin binding domain connected by a linker sequence. The C5-binding protein has a theoretical molecular weight of approximately 11.9 kDa and a theoretical isoelectric point (pI) of 4.42. The amino acid sequence of the C5-binding protein, is as follows:

TABLE 1
Amino Acid Sequence of C5-binding protein

SEQ ID NO. 1
AEAKYAKEVLEAWDEIDRLPNLTIEQWLAFINKLDDDPSQSSELLS
EAKKLSESQAPKVEGSLAEAKEAANAELDSYGVSDFYKRLIDKAKT
VEGVEALKDAILAALP

The C5-binding protein binds to the human complement protein C5 with high affinity and inhibits complement pathway function and terminal complement activation.

The C5-binding protein can be produced using standard methods known in the art for the production of recombinant proteins. In some embodiments the C5-binding protein may be produced synthetically, for example by solid phase synthesis. In some embodiments the C5-binding protein can be expressed from a recombinant nucleic acid molecule that encodes SEQ ID NO. 1 in any suitable host cell(s). In some embodiments the host cells are mammalian cells. In some embodiments the host cells are plant cells. In some embodiments the host cells are insect cells. In some embodiments the host cells are yeast cells. In some embodiments the host cells are bacterial cells. In some embodiments the C5-binding protein may be produced by expression in Escherichia coli cells. In some embodiments a nucleotide sequence that encodes SEQ ID NO. 1 may be codon optimized for expression in the selected host cell type. For example, in some embodiments a nucleotide sequence that encodes SEQ ID NO. 1 may be codon optimized for bacterial expression. Once expressed, the active agent can be purified using any suitable method known in the art, including, but not limited to, chromatography-based methods.

Further details of the generation of the active agent are provided in WO2015/028558 and U.S. Pat. No. 9,994,626, the contents of which are hereby incorporated by reference.

### Pharmaceutical Compositions

The active agent of the present invention may be formulated in a pharmaceutical composition that comprises the active agent and one or more additional pharmaceutically-acceptable components.

In some embodiments, the pharmaceutical composition may comprise one or more carriers, diluents, excipients, or other additives. For example, in some embodiments the pharmaceutical composition comprises one or more stabilizing agents, one or more buffers, one or more pH adjusting agents, one or more surfactants, and/or one or more diluents (e.g., water, physiological saline). In some embodiments the pharmaceutical composition does not comprise a preservative.

In some embodiments, the pharmaceutical composition comprises histidine. In some embodiments, the pharmaceutical composition comprises arginine. In some embodiments, the pharmaceutical composition comprises polysorbate 20. In some embodiments, the pharmaceutical composition comprises histidine, arginine, polysorbate 20, and water (water for injection). In some embodiments, the pharmaceutical composition comprises the active agent, about 20 mM histidine, about 150 mM arginine, and about 0.05% polysorbate 20 (v/v), in water (e.g., water for injection).

In some embodiments the pH of the composition is between about 5.0 and about 9.0. In some embodiments the pH of the composition is between about 6.0 and about 8.0. In some embodiments the pH of the composition is between about 6.5 and about 7.5. In some embodiments the pH of the composition is about 7.0.

In some embodiments, the pharmaceutical composition comprises the active agent, about 20 mM histidine, about 150 mM arginine, and about 0.05% polysorbate 20 (v/v), in water and has a pH of about 7.0.

In some embodiments, the pharmaceutical composition comprises the active agent at a concentration of about 50 mg/mL to 150 mg/mL. In some embodiments, the pharmaceutical composition comprises the active agent at a concentration of about 60 mg/mL to 140 mg/mL. In some embodiments, the pharmaceutical composition comprises the active agent at a concentration of about 70 mg/mL to 130 mg/mL. In some embodiments, the pharmaceutical composition comprises the active agent at a concentration of about 80 mg/mL to 120 mg/mL. In some embodiments, the pharmaceutical composition comprises the active agent at a concentration of about 90 mg/mL to 110 mg/mL. In some embodiments, the pharmaceutical composition comprises the active agent at a concentration of about 100 mg/mL.

In some embodiments, the pharmaceutical composition comprises the active agent at a concentration of about 100 mg/mL, about 20 mM histidine, about 150 mM arginine, and about 0.05% polysorbate 20 (v/v), in water and has a pH of about 7.0

In some embodiments, the pharmaceutical composition is stable when stored at from about 2° C. to about 8° C. In some embodiments, the pharmaceutical composition is stable when stored at about 2° C. to about 8° C. for a period of about 1 week, or a period of about 2 weeks, or a period of about 1 month, or a period of about 2 months, or a period of about 3 months, or a period of about 6 months, or a period of about 9 months, or a period of about 12 months, or a period of about 18 months, or a period of about 24 months, or a period of about 30 months, or a period of about 36 months, or more.

In some embodiments, the pharmaceutical composition is stable when stored at from about 15° C. to about 25° C. In some embodiments, the pharmaceutical composition is stable when stored at about 15° C. to about 25° C. for a period of about 12 hours, or a period of about 24 hours, or a period of about 36 hours, or a period of about 48 hours, or a period of about 3 days, or a period of about 4 days, or a period of about 5 days, or a period of about 6 days, or a period of about 1 week, or a period of more than 1 week.

### Methods of Administration

The present invention provides various methods that involve the administration of the active agent described herein (the C5-binding protein) to subjects. In such methods, the active agent is administered to a subject in a pharmaceutical composition (details of which are also described herein). Thus, whenever any description is provided herein that refers the active agent (for example in relation to methods of administration, amounts to be administered, dosage/administration schedules, routes of administration, and the like), such description is to be understood as also referring to a pharmaceutical composition comprising the active agent.

In some embodiments the subjects to whom the active agents are administered are mammalian subjects. In some embodiments the subjects to whom the active agents are administered are human subjects.

In some embodiments the methods described herein are performed to achieve certain biological effects in the subject. In some embodiments such methods involve administration of certain amounts of the active agent. In some embodiments such methods involve administration of a certain number of doses of the active agent. In some embodiments such methods involve administration of the active agent according to a certain dosing schedule. In some embodiments such methods involve administration of the active agent using certain delivery routes.

In some embodiments the present invention provides methods that achieve certain biological effects. For example, in some embodiments the present invention provides methods that inhibit C5 activity in a subject. In some embodiments the present invention provides methods that inhibit the cleavage of C5 to C5a and C5b in a subject. In some embodiments the present invention provides methods that inhibit activation of the terminal complement pathway in a subject. In some embodiments the present invention provides methods that reduce the amount of one or more components of the terminal complement pathway in a subject. In some embodiments the present invention provides methods that reduce the amount of a terminal complement pathway component selected from C5a, C5b, C6, C7, C8, and C9 in a subject. In some embodiments the present invention provides methods that reduce the amount of C5a in a subject. In some embodiments the present invention provides methods that reduce the amount of C5b in a subject.

In some embodiments the present invention provides methods that reduce the amount of free C5 in a subject. In some embodiments the present invention provides methods that reduce the serum free C5 concentration to about 1.0 micrograms/mL or less. In some embodiments the present invention provides methods that reduce the serum free C5 concentration to about 0.9 micrograms/mL or less. In some embodiments the present invention provides methods that reduce the serum free C5 concentration to about 0.8 micrograms/mL or less. In some embodiments the present invention provides methods that reduce the serum free C5 concentration to about 0.7 micrograms/mL or less. In some embodiments the present invention provides methods that reduce the serum free C5 concentration to about 0.6 micrograms/mL or less. In some embodiments the present invention provides methods that reduce the serum free C5 concentration to about 0.5 micrograms/mL or less. In some embodiments the present invention provides methods that reduce the serum free C5 concentration to about 0.4 micrograms/mL or less. In some embodiments the present invention provides methods that reduce the serum free C5 concentration to about 0.3 micrograms/mL or less. In some embodiments the present invention provides methods that reduce the serum free C5 concentration to about 0.2 micrograms/mL or less. In some embodiments the present invention provides methods that reduce the serum free C5 concentration to about 0.1 micrograms/mL or less. In some embodiments the present invention provides methods that reduce the serum free C5 concentration to about 0.05 micrograms/mL or less. In some embodiments the present invention provides methods that reduce the serum free C5 concentration to about 0.01 micrograms/mL or less. In some embodiments the present invention provides methods that reduce the serum free C5 concentration to about 0.005 micrograms/mL or less. In some embodiments the present invention provides methods that reduce the serum free C5 concentration to about 0.001 micrograms/mL or less.

In some embodiments the present invention provides methods that involve administration of certain amounts of the active agent of the present invention to subjects. These amounts of the active agent are administered to subjects in a pharmaceutical composition.

In some embodiments such amounts are administered to a subject only once—i.e., one dose of a certain amount of the active agent is administered to a subject. In other embodiments such amounts are administered to subjects more than once—i.e., a series of multiple (two or more) doses is administered to a subject—as further described below.

In those embodiments where a series of multiple doses is administered to a subject, the doses may be administered on a certain dosing schedule—for example with certain specified time intervals between individual doses in the series of doses. In some embodiments the amount administered to a subject in each dose within the series of doses is the same (i.e., each dose within the series of doses contains the same amount of the active agent). In other embodiments the amount administered to the subject in each dose within the series of doses is the not the same (i.e., individual doses within the series of doses may contain differing amounts of the active agent).

In some embodiments the amount of the active agent that is administered to a subject is about 2 mg. In some embodiments the amount administered is about 10 mg. In some embodiments the amount administered is about 20 mg. In some embodiments the amount administered is about 30 mg. In some embodiments the amount administered is about 40 mg. In some embodiments the amount administered is about 50 mg. In some embodiments the amount administered is about 60 mg. In some embodiments the amount administered is about 70 mg. In some embodiments the amount administered is about 80 mg. In some embodiments the amount administered is about 90 mg. In some embodiments the amount administered is about 100 mg. In some embodiments the amount administered is about 110 mg. In some embodiments the amount administered is about 120 mg. In some embodiments the amount administered is about 130 mg. In some embodiments the amount administered is about 140 mg. In some embodiments the amount administered is about 150 mg. In some embodiments the amount administered is about 160 mg. In some embodiments the amount administered is about 170 mg. In some embodiments the amount administered is about 180 mg. In some embodiments the amount administered is about 190 mg. In some embodiments the amount administered is about 200 mg. In some embodiments the amount administered is about 210 mg. In some embodiments the amount administered is about 220 mg. In some embodiments the amount administered is about 230 mg. In some embodiments the amount administered is about 240 mg. In some embodiments the amount administered is about 250 mg. In some embodiments the amount administered is about 260 mg. In some embodiments the amount administered is about 270 mg. In some embodiments the amount administered is about 280 mg. In some embodiments the amount administered is about 290 mg. In some embodiments the amount administered is about 300 mg. In some embodiments the amount administered is about 325 mg. In some embodiments the amount administered is about 350 mg. In some embodiments the amount administered is about 375 mg. In some embodiments the amount administered is about 400 mg. In some embodiments the amount administered is about 425 mg. In some embodiments the amount administered is about 450 mg. In some embodiments the amount administered is about 475 mg. In some embodiments the amount administered is about 500 mg. In some embodiments the amount administered is about 600 mg. In some embodiments the amount administered is about 700 mg. In some embodiments the amount administered is about 800 mg. In some embodiments the amount administered is about 900 mg. In some embodiments the amount administered is about 1000 mg. In some embodiments the amount administered is up to about 2000 mg. In some embodiments the amount administered is up to about 3000 mg. In some embodiments the amount administered is up to the “no observed adverse events level” (NOAEL). These amounts can also serve as endpoints for a range of amounts to be administered—in any combinations. For example, an amount of from about 2 to about 1000 mg, or from about 100 to about 500 mg, and the like.

In some embodiments the amount administered is from about 2 mg to about 500 mg. In some embodiments the amount administered is from about 10 mg to about 500 mg. In some embodiments the amount administered is from about 30 mg to about 500 mg. In some embodiments the amount administered is from about 50 mg to about 500 mg. In some embodiments the amount administered is from about 100 mg to about 500 mg. In some embodiments the amount administered is from about 100 mg to about 400 mg. In some embodiments the amount administered is from about 100 mg to about 300 mg. In some embodiments the amount administered is from about 100 mg to about 200 mg. In some embodiments the amount administered is from about 150 mg to about 500 mg. In some embodiments the amount administered is from about 150 mg to about 400 mg. In some embodiments the amount administered is from about 150 mg to about 300 mg. In some embodiments the amount administered is from about 150 mg to about 200 mg. In some embodiments the amount administered is from about 30 mg to about 300 mg. In some embodiments the amount administered is from about 30 mg to about 200 mg. In some embodiments the amount administered is from about 30 mg to about 150 mg. In some embodiments the amount administered is from about 30 mg to about 100 mg. In some embodiments the amount administered is from about 50 mg to about 300 mg. In some embodiments the amount administered is from about 50 mg to about 200 mg. In some embodiments the amount administered is from about 50 mg to about 150 mg. In some embodiments the amount administered is from about 50 mg to about 100 mg. In some embodiments the amount administered is from about 75 mg to about 300 mg. In some embodiments the amount administered is from about 75 mg to about 200 mg. In some embodiments the amount administered is from about 75 mg to about 150 mg. In some embodiments the amount administered is from about 75 mg to about 100 mg. The amounts above are described as absolute amounts in milligrams, based on amounts that may be administered to an average adult human subject of about 70 kg in weight. These absolute amounts in mg may be converted to amounts in mg/kg bodyweight by multiplying the amounts provided above by a factor of about 0.015 (i.e., an amount of 1 mg above corresponds to an amount of 0.015 mg/kg bodyweight).

For example, in some embodiments the amount administered may be about 0.03 mg/kg bodyweight. In some embodiments the amount administered may be about 0.14 mg/kg bodyweight. In some embodiments the amount administered may be about 0.43 mg/kg bodyweight. In some embodiments the amount administered may be about 1.4 mg/kg bodyweight. In some embodiments the amount administered may be about 2.2 mg/kg bodyweight. In some embodiments the amount administered may be about 2.7 mg/kg bodyweight. In some embodiments the amount administered may be about 4.3 mg/kg bodyweight. In some embodiments the amount administered may be about 5.4 mg/kg bodyweight. In some embodiments the amount administered may be about 6.75 mg/kg bodyweight. These amounts can also serve as endpoints for a range of amounts to be administered—in any combinations. For example, an amount of from about 0.03 mg/kg to about 6.75 mg/kg, or an amount of from about 1.4 mg/kg to about 6.75 mg/kg, or an amount of from about 1.4 mg/kg to about 4.3 mg/kg, and the like.

In some embodiments the above amounts of the active agent are administered to subjects once—as a single dose. However, more typically, the above amounts of the active agent are administered to subjects more than once—i.e., in a series of multiple (two or more) doses. The amount of the active agent in each individual dose within such a series of multiple doses may be any of the amounts described above.

In some embodiments a series of 2 or more doses is administered to a subject. In some embodiments a series of 3 or more doses is administered to a subject. In some embodiments a series of 4 or more doses is administered to a subject. In some embodiments a series of 5 or more doses is administered to a subject. In some embodiments a series of 6 or more doses is administered to a subject. In some embodiments a series of 7 or more doses is administered to a subject. In some embodiments a series of 8 or more doses is administered to a subject. In some embodiments a series of 9 or more is administered to a subject. In some embodiments a series of 10 or more is administered to a subject.

In some embodiments the individual doses in a series of multiple doses are administered at time interval of about every 2 days (Q2D). In some embodiments the individual doses in a series of multiple doses are administered at an interval of about every 3 days (Q3D). In some embodiments the individual doses in a series of multiple doses are administered at an interval of about every 4 days (Q4D). In some embodiments the individual doses in a series of multiple doses are administered at an interval of about every 5 days (Q5D). In some embodiments the individual doses in a series of multiple doses are administered at an interval of about every 6 days (Q6D). In some embodiments the individual doses in a series of multiple doses are administered at an interval of about every 7 days (Q7D/QW). In some embodiments the individual doses in a series of multiple doses are administered to a subject at an interval of about every 8 days (Q8D). In some embodiments the individual doses in a series of multiple doses are administered to a subject at an interval of about every 9 days (Q9D). In some embodiments the individual doses in a series of multiple doses are administered to a subject at an interval of about every 10 days (Q10D). In some embodiments the individual doses in a series of multiple doses are administered to a subject at an interval of about every 11 days (Q11D). In some embodiments the individual doses in a series of multiple doses are administered to a subject at an interval of about every 12 days (Q12D). In some embodiments the individual doses in a series of multiple doses are administered to a subject at an interval of about every 13 days (Q13D). In some embodiments the individual doses in a series of multiple doses are administered to a subject at an interval of about every 14 days (Q14D/Q2W).

In some of those embodiments where a series of multiple doses is administered to a subject, the doses are administered in two phases: an initial “induction” phase (which may also be referred to as a “loading” phase) followed by a subsequent “maintenance” phase. The doses administered during the induction phase may be referred to as induction doses. Similarly, the doses administered during the maintenance phase may be referred to as maintenance doses.

In some embodiments the frequency with which the individual doses are administered to the subject is greater during the induction phase as compared to the maintenance phase. Stated another way, the time interval between administration of the individual doses is less during the induction phase as compared to the maintenance phase. For example, in one embodiment individual doses may be administered to a subject about every 2 days (Q2D) during the induction phase and then about every week (QW/Q7D) during the maintenance phase. Similarly, in one embodiment individual doses may be administered to a subject about every 3 days (Q3D) during the induction phase and then about every week (QW/Q7D) during the maintenance phase. In other embodiments individual doses may be administered to a subject at any of the intervals/frequencies set forth above during the induction phase at any of the intervals/frequencies set forth above during the maintenance phase, provided that the time intervals between doses are less during the induction phase than during the maintenance phase.

In some embodiments the amount of active agent administered in an individual dose during the induction phase is greater than the amount administered in an individual dose during the maintenance phase. For example, in some embodiments individual doses administered during the induction phase contain about 1.5 times, or about double, or about triple, or about quadruple, or about 5 times the amount of active agent administered in an individual dose during the maintenance phase. In some such embodiments the frequency with which the individual doses are administered to the subject is the same during the induction phase as compared to the maintenance phase—i.e., the amount administered in an individual dose is higher during the induction phase than the maintenance phase, but the frequency of dosing is the same.

In some embodiments both the amount administered in an individual dose and the frequency with which individual doses are administered is greater during the induction phase as compared to the maintenance phase.

The following Table includes non-limiting examples of methods according to the present invention that include both an induction phase and a maintenance phase for administration of the active agent. Numerous other examples of methods according to the present invention that include both an induction phase and a maintenance phase are provided elsewhere herein, including in the Examples section of this patent specification.

TABLE 2
Examples of Protocols Having Induction & Maintenance Phases

| Phase | Day | Example Protocols | | | | | |
|---|---|---|---|---|---|---|---|
| Induction | Day 1 | 300 mg | 450 mg | 150 mg | 150 mg | 150 mg | 150 mg |
| Phase | Day 3 | — | — | — | 150 mg | — | 150 mg |
| | Day 4 | — | — | 150 mg | — | 150 mg | — |
| | Day 5 | — | — | — | 150 mg | — | 150 mg |
| Maintenance | Day 8 | 150 mg | 150 mg | 150 mg | 150 mg | 300 mg | 300 mg |
| Phase | Day 15 | 150 mg | 150 mg | 150 mg | 150 mg | 300 mg | 300 mg |
| | Day 22 | 150 mg | 150 mg | 150 mg | 150 mg | 300 mg | 300 mg |
| | Day 29 | 150 mg | 150 mg | 150 mg | 150 mg | 300 mg | 300 mg |

In some embodiments the total duration of the induction phase is about 1 week. In some embodiments the total duration of the induction phase is about 2 weeks. In some embodiments the total duration of the induction phase is about 3 weeks.

The total duration of the maintenance phase can be as long as desired, for example as long as the subject is experiencing benefit from the administration method, including indefinitely. In some embodiments the total duration of the maintenance phase is about 4 weeks. In some embodiments the total duration of the maintenance phase is about 2 months. In some embodiments the total duration of the maintenance phase is about 3 months. In some embodiments the total duration of the maintenance phase is about 6 months. In some embodiments the total duration of the maintenance phase is about 9 months. In some embodiments the total duration of the maintenance phase is about 1 year, or more.

In some embodiments, the active agent is administered at an amount and/or frequency effective to reduce the serum free C5 concentration to about 0.5 micrograms/mL or less. In some embodiments, the active agent is administered at an amount and/or frequency effective to reduce the serum free C5 concentration to about 0.1 micrograms/mL or less. In some embodiments, the active agent is administered at an amount and/or frequency effective to reduce the serum free C5 concentration to about 0.05 micrograms/mL or less. In some embodiments, the active agent is administered at an amount and/or frequency effective to reduce the serum free C5 concentration to about 0.01 micrograms/mL or less. In some embodiments, the active agent is administered at an amount and/or frequency effective to reduce the serum free C5 concentration to about 0.005 micrograms/mL or less. In some embodiments, the active agent is administered at an amount and/or frequency effective to reduce the serum free C5 concentration to about 0.001 micrograms/mL or less.

In some embodiments, the active agent is administered at an amount and/or frequency effective to reduce the serum free C5 concentration by about 90%, or by at least 90%, as compared to the baseline serum free C5 concentration prior to administration of the active agent. In some embodiments, the active agent is administered at an amount and/or frequency effective to reduce the serum free C5 concentration by about 95%, or by at least 95%, as compared to the baseline serum free C5 concentration prior to administration of the active agent. In some embodiments, the active agent is administered at an amount and/or frequency effective to reduce the serum free C5 concentration by about 96%, or by at least 96%, as compared to the baseline serum free C5 concentration prior to administration of the active agent. In some embodiments, the active agent is administered at an amount and/or frequency effective to reduce the serum free C5 concentration by about 97%, or by at least 97%, as compared to the baseline serum free C5 concentration prior to administration of the active agent. In some embodiments, the active agent is administered at an amount and/or frequency effective to reduce the serum free C5 concentration by about 98%, or by at least 98%, as compared to the baseline serum free C5 concentration prior to administration of the active agent. In some embodiments, the active agent is administered at an amount and/or frequency effective to reduce the serum free C5 concentration by about 99%, or by at least 99%, as compared to the baseline serum free C5 concentration prior to administration of the active agent. In some embodiments, the active agent is administered at an amount and/or frequency effective to reduce the serum free C5 concentration by about 99.5%, or by at least 99.5%, as compared to the baseline serum free C5 concentration prior to administration of the active agent. In some embodiments, the active agent is administered at an amount and/or frequency effective to reduce the serum free C5 concentration by about 99.9%, or by at least 99.9%, as compared to the baseline serum free C5 concentration prior to administration of the active agent.

In some embodiments, the active agent is administered at an amount and/or frequency effective to achieve a Cₘₐₓ of the active agent of from about 0.1 micromolar to about 10.0 micromolar. For example, in some embodiments, the active agent is administered at an amount and/or frequency effective to achieve a Cₘₐₓ of the active agent of from about 1.0 micromolar to about 5.0 micromolar. In some embodiments, the active agent is administered at an amount and/or frequency effective to achieve a Cₘₐₓ of the active agent of about 1.0 micromolar, or about 2.0 micromolar, or about 3.0 micromolar, or about 4.0 micromolar, or about 5.0 micromolar, or about 6.0 micromolar, or about 7.0 micromolar, or about 8.0 micromolar, or about 9.0 micromolar, or about 10.0 micromolar.

In some embodiments, the active agent is administered at an amount and/or frequency effective to achieve a C_{trough} of the active agent of from about 0.1 to about 10 micromolar. In some embodiments, the active agent is administered at an amount and/or frequency effective to achieve a C_{trough} of the active agent of from about 0.5 to about 5.0 micromolar. In some embodiments, the active agent is administered at an amount and/or frequency effective to achieve a C_{trough} of 0.5 micromolar or above. In some embodiments, the active agent is administered at an amount and/or frequency effective to achieve a C_{trough} of 0.6 micromolar or above. In some embodiments, the active agent is administered at an amount and/or frequency effective to achieve a C_{trough} of 0.7 micromolar or above. In some embodiments, the active agent is administered at an amount and/or frequency effective to achieve a C_{trough} of 0.8 micromolar or above. In some embodiments, the active agent is administered at an amount and/or frequency effective to achieve a C_{trough} of 0.9 micromolar or above. In some embodiments, the active agent is administered at an amount and/or frequency effective to achieve a C_{trough} of 1.0 micromolar or above. In some embodiments, the active agent is administered at an amount and/or frequency effective to achieve a C_{trough} of 1.1 micromolar or above. In some embodiments, the active agent is administered at an amount and/or frequency effective to achieve a C_{trough} of 1.2 micromolar or above. In some embodiments, the active agent is administered at an amount and/or frequency effective to achieve a C_{trough} of 1.3 micromolar or above. In some embodiments, the active agent is administered at an amount and/or frequency effective to achieve a C_{trough} of 1.4 micromolar or above. In some embodiments, the active agent is administered at an amount and/or frequency effective to achieve a C_{trough} of 1.5 micromolar or above. In some embodiments, the active agent is administered at an amount and/or frequency effective to achieve a C_{trough} of 1.6 micromolar or above. In some embodiments, the active agent is administered at an amount and/or frequency effective to achieve a C_{trough} of 1.7 micromolar or above. In some embodiments, the active agent is administered at an amount and/or frequency effective to achieve a C_{trough} of 1.8 micromolar or above. In some embodiments, the active agent is administered at an amount and/or frequency effective to achieve a C_{trough} of 1.9 micromolar or above. In some embodiments, the active agent is administered at an amount and/or frequency effective to achieve a C_{trough} of 2.0 micromolar or above. In some embodiments, the active agent is administered at an amount and/or frequency effective to achieve a C_{trough} of 1.0 micromolar or above. In some embodiments, the active agent is administered at an amount and/or frequency effective to achieve a C_{trough} of 2.5 micromolar or above. In some embodiments, the active agent is administered at an amount and/or frequency effective to achieve a C_{trough} of 3.0 micromolar or above. In some embodiments, the active agent is administered at an amount and/or frequency effective to achieve a C_{trough} of 3.5 micromolar or above. In some embodiments, the active agent is administered at an amount and/or frequency effective to achieve a C_{trough} of 4.0 micromolar or above. In some embodiments, the active agent is administered at an amount and/or frequency effective to achieve a C_{trough} of 4.5 micromolar or above. In some embodiments, the active agent is administered at an amount and/or frequency effective to achieve a C_{trough} of 5.0 micromolar or above.

In some embodiments, the active agent is administered at an amount and/or frequency effective to achieve a Tₘₐₓ of the active agent of about 24-72 hours. For example, in some embodiments, the active agent is administered at an amount and/or frequency effective to achieve a Tₘₐₓ of the active agent of about 24 hours. In some embodiments, the active agent is administered at an amount and/or frequency effective to achieve a Tₘₐₓ of the active agent of about 48 hours. In some embodiments, the active agent is administered at an amount and/or frequency effective to achieve a Tₘₐₓ of the active agent of about 72 hours.

In some embodiments, the active agent is administered at an amount and/or frequency effective to achieve a T_{1/2} of the active agent of about 300-400 hours. For example, in some embodiments, the active agent is administered at an amount and/or frequency effective to achieve a T_{1/2} of the active agent of about 350 hours.

In some embodiments the active agents are administered to subjects at any of the amounts and/or according to any of the dosing schedules described in the Examples section of this patent disclosure.

In some embodiments, the active agents of the present invention are administered to subjects parenterally. Parenteral routes of administration include intravenous, intramuscular, intraperitoneal, intrathecal, and subcutaneous administration. In preferred embodiments, the pharmaceutical compositions of the present invention are administered subcutaneously. In some embodiments, the pharmaceutical compositions of the present invention are administered as a low volume subcutaneous injection. In some embodiments, the pharmaceutical compositions of the present invention are administered as a subcutaneous injection having a volume of 2.5 mL or less. In some embodiments, the pharmaceutical compositions of the present invention are administered as a subcutaneous injection having a volume of 2.25 mL or less. In some embodiments, the pharmaceutical compositions of the present invention are administered as a subcutaneous injection having a volume of 2.0 mL or less. In some embodiments, the pharmaceutical compositions of the present invention are administered as a subcutaneous injection having a volume of 1.75 mL or less. In some embodiments, the pharmaceutical compositions of the present invention are administered as a subcutaneous injection having a volume of 1.5 mL or less. In some embodiments, the pharmaceutical compositions of the present invention are administered as a subcutaneous injection having a volume of 1 mL or less. In some embodiments, the pharmaceutical compositions of the present invention are administered as a subcutaneous injection having a volume of 0.75 mL or less. In some embodiments, the pharmaceutical compositions of the present invention are administered as a subcutaneous injection having a volume of 0.5 mL or less. In some embodiments, the pharmaceutical compositions of the present invention are administered as a subcutaneous injection having a volume of about 2.5 mL. In some embodiments, the pharmaceutical compositions of the present invention are administered as a subcutaneous injection having a volume of about 2.25 mL. In some embodiments, the pharmaceutical compositions of the present invention are administered as a subcutaneous injection having a volume of about 2.0 mL. In some embodiments, the pharmaceutical compositions of the present invention are administered as a subcutaneous injection having a volume of about 1.75 mL. In some embodiments, the pharmaceutical compositions of the present invention are administered as a subcutaneous injection having a volume of about 1.5 mL. In some embodiments, the pharmaceutical compositions of the present invention are administered as a subcutaneous injection having a volume of about 1 mL. In some embodiments, the pharmaceutical compositions of the present invention are administered as a subcutaneous injection having a volume of about 0.75 mL. In some embodiments, the pharmaceutical compositions of the present invention are administered as a subcutaneous injection having a volume of about 0.5 mL.

In some embodiments, the active agents of the present invention are administered to subjects by a healthcare provider. In some embodiments, the active agents of the present invention are administered to a subject by someone who is not a healthcare provider, for example the subject themselves, a family member, a caregiver, and the like. In some embodiments, the active agents of the present invention are self-administered. In some embodiments, the active agents of the present invention are administered using an autoinjector.

In some embodiments, the active agent of the present invention is provided in autoinjector device. In some embodiments, the present invention provides an autoinjector device comprising a pharmaceutical composition according to the present invention. In some of such embodiments, the amount of the pharmaceutical composition or active agent in the autoinjector device can be any of the amounts described herein, for example an amount suitable for administration as a single dose (such as a single dose in a series of multiple doses) or an amount suitable for administration of more than one dose. In some embodiments, the present invention provides an autoinjector device comprising a pharmaceutical composition comprising a C5-binding protein at a concentration of about 100 mg/mL.

In some embodiments, the present invention provides a kit containing a pharmaceutical composition comprising the active agent of the present invention and instructions for the use thereof. In some embodiments, the present invention provides a kit containing a pharmaceutical composition comprising the active agent of the present invention in an autoinjector device and instructions for the use thereof. In some embodiments, the present invention provides a kit containing a pharmaceutical composition comprising the active agent of the present invention, an autoinjector device, and instructions for the use thereof.

In some embodiments the active agent and pharmaceutical compositions provided herein may be administered to a subject without inducing severe adverse events (AEs) or without inducing serious adverse events (SAEs).

Embodiments of the present disclosure can be further defined by reference to the following non-limiting examples. It will be apparent to those skilled in the art that many modifications, both to materials and methods, can be practiced without departing from the scope of the present disclosure.

### EXAMPLES

### Example 1

### Non-Clinical Pharmacology

The C5-binding protein of the present invention was expressed in Escherichia coli from a recombinant nucleic acid encoding SEQ ID NO. 1, purified, and formulated in a phosphate buffered saline (PBS).

The specificity and affinity of binding of the C5-binding protein (73.46 mg/mL in PBS) to human C5 in vitro was determined by measuring the kinetics of its binding to human C5 as compared to its binding to human C3, C4, and human IgG using surface plasmon resonance methods. C3 and C4 are the 2 proteins most closely related to C5, sharing approximately 30% overall protein sequence identity to human C5 (Wetsel et al, 1988, “Molecular Analysis of Human Complement Component C5: Localization of the Structural Gene to Chromosome 9:” Biochemistry; Vol. 27(5): pp. 1474-1482), and IgG is the original ligand of the affibody scaffold from which the affibody portion of the C5-binding protein was engineered. The C5-binding protein was observed to bind to the human C5 protein with an affinity of approximately 0.38 nM (K_{D}), while no physiologically relevant binding was observed for human C3, C4, or IgG. The results indicate a selectivity of the C5-binding protein for human C5 over C3, C4, and IgG of at least 4000-fold.

Complement classical pathway hemolytic assays with antibody-sensitized sheep erythrocytes were performed with the objective of evaluating the C5-binding protein activity in blocking complement C5 activation. The C5-binding protein (0.0625, 0.125, 0.25, 0.5, 1, and 2 μM) was incubated ex vivo with human serum (a serum pool from multiple healthy donors) and the % of hemolysis was determined. The hemolytic activity reached>95% inhibition at the concentration of 0.5 μM the C5-binding protein, indicating that this concentration may be sufficient for complement blockade in human serum. Hemolytic inhibition data is shown in FIG. 1.

### Example 2

### Phase 1 Single Ascending Dose and Multiple Ascending Dose Study to Evaluate the Safety, Tolerability, Pharmacokinetics, and Pharmacodynamics of a C5-Binding Protein in Healthy Participants

This is a single-blind, placebo-controlled Phase 1 study of the safety, tolerability, pharmacokinetics (PK), and pharmacodynamics (PD) of single and multiple ascending subcutaneous (SC) doses of a pharmaceutical composition comprising the C5-binding protein of the present invention (which may be referred to herein as “the study drug”) in healthy human subjects (referred to herein as study participants).

TABLE 3
Objectives and Endpoints

| | |
|---|---|
| Objectives | Endpoints |

Primary

| | |
|---|---|
| To assess the safety and | type, seriousness, and incidence |
| tolerability of the C5-binding | of adverse events (AEs) |
| protein in healthy participants | vital signs |
| following single and | clinical laboratory values |
| repeated administration | electrocardiogram (ECG) |

Secondary

| | |
|---|---|
| To characterize the | measurement of free C5 concentrations |
| pharmacodynamic (PD) | measurement of total C5 concentrations |
| properties of the C5-binding | |
| protein following single and | |
| repeated administration to | |
| healthy participants | |
| To evaluate the immunogen- | occurrence of anti-drug antibodies |
| icity of single and multiple | (ADAs) against the C5-binding protein |
| doses of the C5-binding | |
| protein | |
| To establish the pharmaco- | PK parameters of the C5-binding protein: |
| kinetic (PK) profile of | half-life (t_{1/2}) |
| the C5-binding protein | maximum plasma concentration (Cₘₐₓ) |
| following subcutaneous | time to maximum plasma concentration |
| (SC) administration | (Tₘₐₓ) |
| | clearance (CL) |
| | clearance (CL) |
| | volume of distribution (Vd) |
| | area under the concentration-time curve |
| | (AUC) |

### Overall Design

This study is a single-blind, placebo-controlled, ascending dose, multi-center study to evaluate the safety, tolerability, PK, and PD of single (Part A) and multiple (Part B) doses of the C5-binding protein in healthy participants.

In both parts of the study, participants enter a 70-day screening period after signing the informed consent form. Eligible participants are enrolled and enter the treatment period (which varies by part of the study, from 1 day to 4 weeks). After the last dose received, study participants enter a 10-week follow-up period.

### Part A

Up to 6 ascending dose cohorts are dosed in a sequential manner in Part A of the study. Eight participants are treated with a single dose of the study drug in each dose cohort (6 participants per dose level receive the C5-binding protein and 2 participants receive matching placebo). For each dose cohort, a sentinel group consisting of 2 participants (1 on the C5-binding protein and 1 on placebo) receive their assigned treatment, complete the assessments scheduled up to 24 hours after dose, and undergo safety review before continuation of enrollment in the dose cohort can occur. No more than 4 participants are dosed in 1 day.

Escalation to the next higher dose level occurs only after completion of a review of clinical safety data by the Safety Review Committee.

### Part B

Up to 5 ascending, multiple dose cohorts are enrolled in a sequential manner in Part B of the study. The multiple dose phase initiates after the Safety Review Committee review the safety data from the fifth cohort of the single dose phase and agreement is reached on the study proceeding.

In each dose cohort in Part B, 12 participants are treated with study drug for 4 weeks (10 participants per cohort receive the C5-binding protein and 2 participants per cohort receive matching placebo). Escalation to the next higher dose level occurs only after completion of a review of clinical safety and available PK data by the Safety Review Committee.

### Number of Participants

Around 48 healthy male and female participants (8 participants in each of 6 dose cohorts) are included in Part A and around 60 healthy male and female participants (12 in each of 5 dose cohorts) in Part B,

### Eligibility Criteria

The inclusion exclusion criteria for the study are as follows:

TABLE 4
Inclusion Criteria

| | |
|---|---|
| 1 | Male and female participants, 18 to 55 years of age. |
| 2 | Able to provide written informed consent. |
| 3 | Body mass index (BMI) of 18.0 to 32.0 kg/m². |
| 4 | Must have been vaccinated against N. meningitidis |
| | and S. pneumoniae with approved vaccine according to |
| | product label. All participants considered eligible for |
| | enrollment are vaccinated according to the following: |
| | Vaccination with Meningococcal Group A, C, W135, and |
| | Y conjugate vaccine + Pneumococcal Polysaccharide |
| | Vaccine at least 28 days prior to receiving the |
| | first dose of the C5-binding protein. |
| | Vaccination with Meningococcal Group B at least 14 days |
| | prior to receiving the first dose of the C5-binding protein. |
| 5 | Male participants are eligible to participate if they |
| | agree to the following during the intervention period |
| | and for at least 105 days after the last dose of study drug: |
| | (a) Refrain from donating fresh unwashed semen, plus, either: |
| | (i) Be abstinent from heterosexual intercourse as their |
| | preferred and usual lifestyle (abstinent on a long |
| | term and persistent basis) and agree to remain abstinent, |
| | or (ii) agree to use a male condom with female partner use |
| | of an additional highly effective contraceptive method with a |
| | failure rate of <1% per year when having sexual intercourse |
| | with a woman of childbearing potential (WOCBP) who is |
| | not currently pregnant. |
| 6 | A female participant is eligible to participate if she is not |
| | pregnant or breastfeeding, and one of the following conditions |
| | applies: (a) she is a woman of nonchildbearing potential (WONCBP), |
| | (b) she is a WOCBP and using a contraceptive method that is highly |
| | effective during the study intervention period and for at |
| | least 105 days after the last dose of study drug. A WOCBP |
| | must have a negative highly sensitive pregnancy test (urine or |
| | serum as required by local regulations) within 24 hours |
| | before the first dose of study intervention. |
| 7 | Is expected to adequately comply with required study |
| | procedures and restrictions. |

TABLE 5
Exclusion Criteria

| | |
|---|---|
| 1 | Clinically significant medical history, |
| | current medical, psychiatric, or any other |
| | condition that, in the opinion of the investigator, |
| | would interfere with the participant's |
| | ability to participate in the study or increase |
| | the risk of participation for that participant. |
| 2 | Clinically significant electrocardiogram (ECG) |
| | finding or laboratory/urinalysis abnormality. |
| 3 | Continuous use of any medication, including |
| | over-the-counter (OTC) drugs and herbal |
| | preparations, within 7 days before the first |
| | study drug administration, with the exception |
| | of hormone replacement therapy, oral contraceptives, |
| | OTC adrenergic nasal spray for relief of nasal |
| | congestion and acetaminophen (paracetamol) |
| | for pain and/or fever relief. |
| 4 | Administration of any other investigational drug |
| | within 90 days (or 5 half-lives of the medication, |
| | whichever is longer) of screening. |
| 5 | Recent or current history of alcohol or |
| | drug abuse within the last 12 months. |
| 6 | Participants that smoke >10 cigarettes per week. |
| 7 | Positive serology for HIV or active infection |
| | with hepatitis B virus or hepatitis C virus. |
| 8 | Pregnant or nursing. |
| 9 | Donation or loss of greater than 400 mL of |
| | blood within 56 days of study enrollment. |
| 10 | History of severe hypersensitivity to any drug, |
| | including penicillin, or to N. meningitidis |
| | or S. pneumoniae vaccines. |
| 11 | History of infection with N. meningitidis. |
| 12 | Evidence of active or latent tuberculosis (TB). |

### Study Intervention(s)

The C5-binding protein (purified following expression in Escherichia coli from a recombinant nucleic acid encoding SEQ ID NO. 1) is provided in a sterile, preservative-free, clear to slightly opalescent liquid formulation comprising 100 mg/mL the C5-binding protein buffered to a target of pH 7.0 in a formulation containing histidine, arginine, polysorbate 20, and Water for Injection(s), and provided in single-dose borosilicate glass vials. The placebo is a 0.9% saline (sodium chloride) solution. The C5-binding protein is stored at 2 to 8° C. and brought to room temperature for dilution prior to use and administration.

TABLE 6
Products for Administration

| | C5-Binding Protein | Placebo |
|---|---|---|
| Type | Biologic | Drug |
| Dose Formulation | Parenteral solution | Sodium chloride injection, |
| | | 0.9% (saline) |
| Unit Dose | 100 mg/mL | Not applicable |
| Strength(s) | | |
| Dosage Level(s) | See Table 3 | |
| Route of | SC | |
| Administration | | |
| Use | Investigational | Placebo comparator |

In Part A, up to 48 healthy participants are enrolled across up to 6 sequential ascending dose cohorts (8 participants/cohort). Participants receive a single dose of the C5-binding protein (2, 10, 30, 100, 150, and 300 mg) or placebo, subcutaneously (SC).

In Part B, up to 60 healthy participants are enrolled across up to 5 cohorts (12 participants/cohort) and receive the C5-binding protein (ascending doses) or placebo, SC, as follows:

Cohorts 1, 2, and 3: 100, 150, or 300 mg, respectively once weekly (QW) (on Days 1, 8, 15, 22, 29)

Cohort 4: 100 mg or 150 mg on Days 1 and 4 (first week) and QW thereafter (Days 8, 15, 22, 29)

Cohort 5: 100 mg or 150 mg on Days 1, 3, and 5 (first week), and QW thereafter (Days 8, 15, 22, 29).

Administered doses of 2, 10, 30, 100, 150 and 300 mg correspond to doses approximate does of 0.03, 0.14, 0.43, 1.4, 2.15, 4.3 mg/kg bodyweight, based on a 70 kg human.

The details of these study interventions are also illustrated in the below table

TABLE 7
Study Intervention Groups

| | Dose Cohort^{a} | | | | | |
|---|---|---|---|---|---|---|
| Study Part | 1 | 2 | 3 | 4 | 5 | 6 |

Part A (single dose)

| | | | | | | |
|---|---|---|---|---|---|---|
| Day 1 | 2 mg | 10 mg | 30 mg | 100 mg | 150 mg | 300 mg |
| | SC | SC | SC | SC | SC | SC |

Part B (multiple dose)^{b}

| | | | | | | |
|---|---|---|---|---|---|---|
| Day 1 (or | 100 mg | 150 mg | 300 mg | 100 mg or 150 mg | 100 mg or 150 mg | — |
| Week 1) | SC | SC | SC | SC on Days 1 | SC on Days 1, 3, | |
| | | | | and 4 | and 5 | |
| Day 8 | 100 mg | 150 mg | 300 mg | 100 mg or 150 mg | 100 mg or 150 mg | — |
| | SC | SC | SC | SC | SC | |
| Day 15 | 100 mg | 150 mg | 300 mg | 100 mg or 150 mg | 100 mg or 150 mg | — |
| | SC | SC | SC | SC | SC | |
| Day 22 | 100 mg | 150 mg | 300 mg | 100 mg or 150 mg | 100 mg or 150 mg | — |
| | SC | SC | SC | SC | SC | |
| Day 29 | 100 mg | 150 mg | 300 mg | 100 mg or 150 mg | 100 mg or 150 mg | — |
| | SC | SC | SC | SC | SC | |

SC = subcutaneous; SRC = safety review committee
^{a}The SRC will approve dose level changes, which may include escalation to the next dose cohort, de-escalation to a lower dose, exploring intermediate doses or higher max dose; continuation, delay or termination of dosing; or repetition or expansion of a cohort; or determination of maximum tolerated dose (MTD) or recommended phase 2 dose (RP2D).
^{b}Dose cohorts may be adjusted for the dose selected or the number of cohorts based on emergent safety, tolerability, pharmacokinetic, or pharmacodynamic data from the study.

### Dose Modification

The dosing schedule may be adjusted to expand a dosing cohort to further evaluate safety, tolerability, PK, and/or PD findings at a given dose level or to remove cohorts or to add cohorts to evaluate additional doses up to the maximum feasible dose, without exceeding the NOAEL exposure (area under the concentration time curve [AUC] or maximum plasma concentration [Cₘₐₓ]) of 48 mg/kg. The study procedures for these additional participant(s)/cohort(s) will be the same as that described for other study participants/cohorts.

For example, Part B cohort 3 may be modified to administer 50 mg or 75 mg or 80 mg or 90 mg or 125 mg or 150 mg or 200 mg once weekly (QW) (on Days 1, 8, 15, 22, 29).

Similarly, Part B cohort 4 may be modified to administer 50 mg or 75 mg or 80 mg or 90 mg or 100 mg or 125 mg or 150 mg or 200 mg on Days 1 and 4 (first week) and QW thereafter (Days 8, 15, 22, 29).

Likewise, Part B cohort 5 may be modified to administer 50 mg or 75 mg or 80 mg or 90 mg or 100 mg or 125 mg or 150 mg or 200 mg on Days 1, 3, and 5 (first week), and QW thereafter (Days 8, 15, 22, 29).

### Safety Review Committee

Throughout the duration of the study, a Safety Review Committee will evaluate all data on an ongoing basis and monitor the overall safety of the study participants at regular meetings.

### Example 3

### Clinical Trial Results

The Phase 1 clinical trial described in Example 2 is ongoing. Dosing in Part A of the study is complete, with a modification of the protocol made to remove Cohort 5. Participants in the remaining cohorts of Part A of the study, which includes administration of single ascending doses of the study drug, have been dosed and the first 4 cohorts have completed their 10-week follow-up period. Pharmacokinetic (PK) and pharmacodynamic (PD) analysis has been performed for Part A Cohorts 1-4 (i.e., 2, 10, 30, and 100 mg single doses). For Part A Cohort 6 (300 mg single dose) analysis of PK and PD data is ongoing. Part A Cohort 5 (150 mg single dose) was omitted from the study (no subjects were dosed for Cohort 5).

A Safety Review Committee (SRC) evaluated data from—all cohorts dosed. Safety was evaluated for each of Cohorts 1-4 before commencing the subsequent higher dose cohort. In each case, the Safety Review Committee determined that, while some mild or moderate adverse events were observed, safety and tolerability were acceptable to progress to the subsequent higher dose cohort. No drug-related serious adverse events were reported in any dosed cohort.

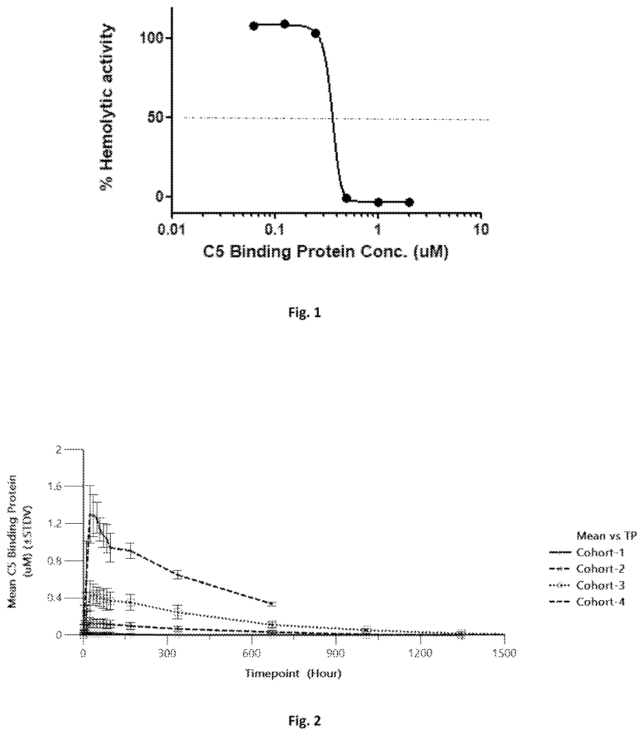

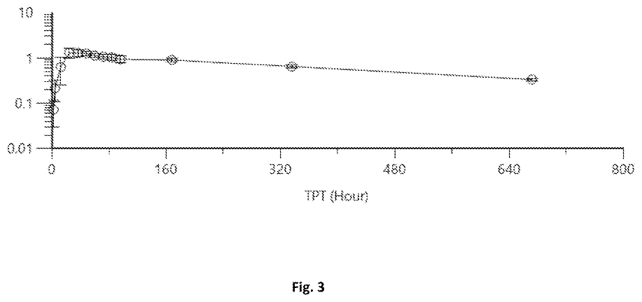

The PK data obtained to-date from Cohorts 1-4 is summarized in Table 8 below. Additional PK data is also shown in FIG. 2 (cohorts 1-4) and FIG. 3 (cohort 4).

TABLE 8

| | | Dose | | | | | | | | |
|---|---|---|---|---|---|---|---|---|---|---|
| Cohort | N | (mg) | R_{sq} | T_{1/2} | Tₘₐₓ | Cₘₐₓ | Tₗₐₛₜ | AUCINF_obs | V | CL |
| 1 | 17 | 2 | 0.97 | NA* | 72 | 0.02 | 168 | 2.13 | 6.27 | 0.09 |
| 2 | 17 | 10 | 0.97 | 242.66 | 24 | 0.13 | 1008 | 55.02 | 5.34 | 0.02 |
| 3 | 16 | 30 | 0.99 | 299.70 | 52 | 0.46 | 1344 | 209.54 | 5.04 | 0.01 |
| 4 | 14 | 100 | 1.00 | 349.78 | 24 | 1.30 | 672 | 621.66 | 6.82 | 0.01 |

*Insufficient data points for determination of T_{1/2}

In Table 8 the data presented is mean data obtained from the number of samples (N) indicated and “R_{sq}” refers to the statistical measure of correlation R-Squared, T_{1/2}: refers to half-life of the study drug in the serum in hours (i.e. the time for the concentration of the study drug in the serum to decrease by 50%), Tₘₐₓ refers to the time in hours after administration for the study drug to reach its maximum concentration in the serum (Cₘₐₓ), Cₘₐₓ refers to the highest concentration of the study drug reached in the serum in micromolar (μM) units, Tₗₐₛₜ refers to the time in hours of the of the last measured concentration of the study drug in the serum, AUCINF_obs refers to the area under the concentration-time curve extrapolated to infinity, V refers to the apparent volume of distribution in Liters (L), and CL refers to clearance in Liters per hour (L/hr).

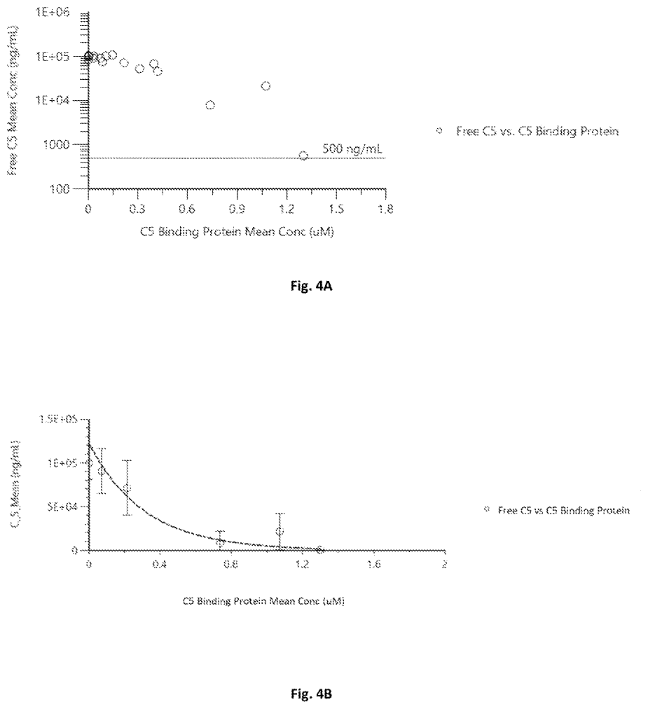

Treatment of subjects with the study drug reduced measured serum free C5 in a concentration-dependent manner (see FIG. 4A and FIG. 4B). Table 9 provides free C5 data for Cohort 4 (100 mg, administered in a 1 mL subcutaneous injection), showing baseline (pre-administration) free C5 levels and free C5 concentrations 24 hours after administration in 6 subjects. Mean free C5 concentrations were reduced to 567 ng/ml (i.e., 0.567 micrograms/ml) representing a mean reduction in free C5 concentrations of 99.4% as compared to the pre-treatment baseline.

TABLE 9

| Participant | BL (ng/mL) | 25 h (ng/mL) | % Reduction |
|---|---|---|---|
| 1 | 91300 | 503 | 99.4% |
| 2 | 91300 | 360 | 99.6% |
| 3 | 84600 | 562 | 99.3% |
| 4 | 94800 | 497 | 99.5% |
| 5 | 100000 | 733 | 99.3% |
| 6 | 135000 | 747 | 99.4% |
| Mean | 99500 | 567 | 99.4% |

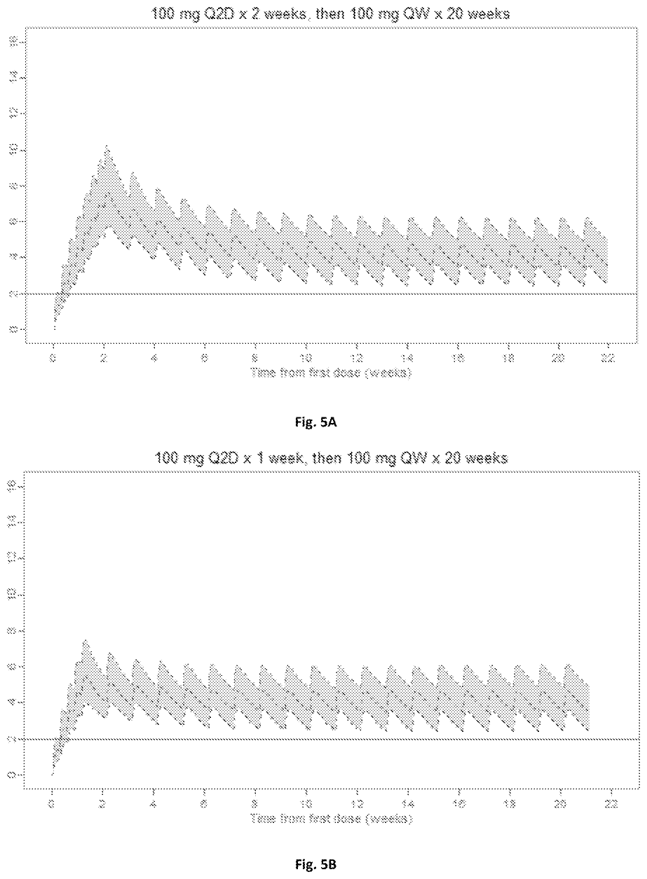

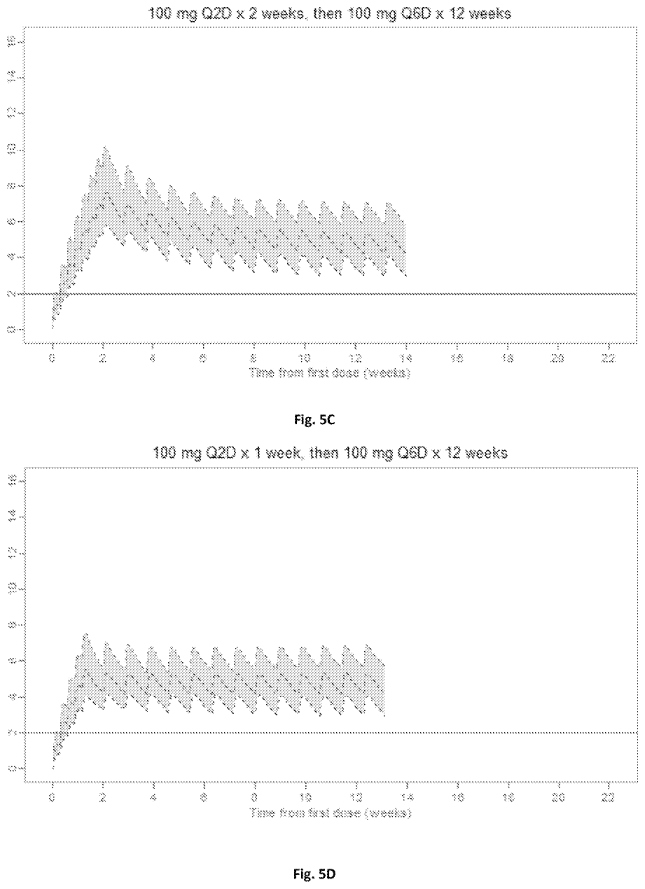

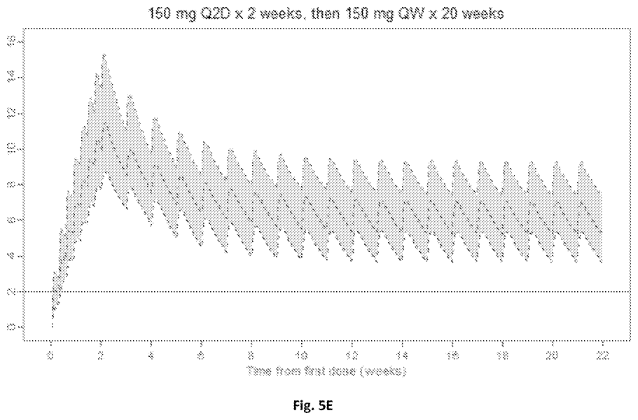

The PK data obtained from Part A Cohorts 1-4 was used as input data for multi-dose pharmacokinetic modeling, which was performed using accepted PK modeling methodologies. The results of these modeling studies for 100 mg and 150 mg doses, administered using various dosing regimens, are provided in FIG. 5 A-E. These results indicate that, in each case, the C5-binding protein rapidly reaches serum concentrations above 2 μM and that serum concentrations are sustained above the 2 μM concentration level even at the troughs (C_{trough}). The 2 μM concentration level is indicated on the graphs in FIG. 5A-E. This is significant because the single dose data described in this Example shows that, even at mean serum concentrations of 1.3 μM of the C5-binding protein, serum free C5 concentrations are reduced by more than 99%, and the hemolytic inhibition data described in Example 1 shows that complete inhibition of hemolysis is achieved at concentrations of the C5-binding protein of 0.5 μM and above. Thus, both the data from the single dose studies (which showed sustained C5-binding protein concentrations over time and good C5 control) and the multi-dose modeling data indicate a favorable PK and PD profile for the C5-binding protein, with weekly, or even less than weekly, administration resulting in effective free C5 reduction over time.

In conclusion, the results of this Phase 1 clinical trial obtained to-date demonstrate that subcutaneous administration of the C5-binding protein provided herein, at doses that can be administered in low volume subcutaneous injections, provides free C5 reduction similar to that achieved by approved anti-C5 therapeutics (as determined by reduction in free C5 concentrations) and has pharmacokinetic properties consistent with providing sustained C5 control over time without the need for frequent (e.g., daily) administration

The present invention is further described by the following claims.

We claim:

1. A method of administering a C5-binding protein to a subject, the method comprising administering to the subject a pharmaceutical composition comprising a C5-binding protein and at least one additional pharmaceutically-acceptable component, wherein:
i. the C5-binding protein comprises SEQ ID NO. 1, and
ii. the pharmaceutical composition is administered to the subject subcutaneously.

2. A method of reducing the amount of free C5 in a subject, the method comprising administering to the subject an effective amount of a pharmaceutical composition comprising a C5-binding protein and at least one additional pharmaceutically-acceptable component, wherein:
i. the C5-binding protein comprises SEQ ID NO. 1, and
ii. the pharmaceutical composition is administered to the subject subcutaneously.

3. The method of claim 2, wherein the serum free C5 concentration is reduced to about 0.5 micrograms/mL or less.

4. The method of claim 2, wherein the serum free C5 concentration is reduced to about 0.1 micrograms/mL or less.

5. A method of inhibiting activation of the terminal complement pathway in a subject, the method comprising administering to the subject an effective amount of a pharmaceutical composition comprising a C5-binding protein and at least one additional pharmaceutically-acceptable component, wherein:
i. the C5-binding protein comprises SEQ ID NO. 1, and
ii. the pharmaceutical composition is administered to the subject subcutaneously.

6. A pharmaceutical composition for use in subcutaneously administering a C5-binding protein to a subject, the pharmaceutical composition comprising:
i. a C5-binding protein comprising SEQ ID NO. 1, and
ii. at least one additional pharmaceutically-acceptable component.

7. A pharmaceutical composition for use in subcutaneously administering an effective amount of a C5-binding protein to a subject to reduce the amount of free C5 in the subject, the pharmaceutical composition comprising:
i. a C5-binding protein comprising SEQ ID NO. 1, and
ii. at least one additional pharmaceutically-acceptable component.

8. The pharmaceutical composition of claim 7, wherein the serum free C5 concentration is reduced to about 0.5 micrograms/mL or less.

9. The pharmaceutical composition of claim 7, wherein the serum free C5 concentration is reduced to about 0.1 micrograms/mL or less.

10. A pharmaceutical composition for use in subcutaneously administering an effective amount of a C5-binding protein to a subject to inhibit activation of the terminal complement pathway, the pharmaceutical composition comprising:
i. a C5-binding protein comprising SEQ ID NO. 1, and
ii. at least one additional pharmaceutically-acceptable component.

11. The method or pharmaceutical composition of any of the preceding claims, wherein the administering comprises administration a single dose of the pharmaceutical composition to the subject.

12. The method or pharmaceutical composition of any of the preceding claims, wherein the administering comprises administration of a series of two or more doses of the pharmaceutical composition to the subject.

13. The method or pharmaceutical composition of claim 12, wherein the two or more doses are administered to the subject at approximately weekly intervals (QW).

14. The method or pharmaceutical composition of any of the preceding claims, wherein the administering comprises administering to the subject: (a) one or more induction doses of the pharmaceutical composition, and (b) one or more maintenance doses of the pharmaceutical composition.

15. The method or pharmaceutical composition of claim 14, wherein the one or more maintenance doses of the pharmaceutical composition are administered at weekly intervals (QW).

16. The method or pharmaceutical composition of any of the preceding claims, wherein the administering comprises administering to the subject: (a) one induction dose of the pharmaceutical composition administered on Day 1, and (b) four or more maintenance doses of the pharmaceutical composition, wherein the first maintenance dose is administered about 1 week after the induction dose, and wherein the second and subsequent maintenance doses are administered at about weekly intervals (QW) thereafter.

17. The method or pharmaceutical composition of any of the preceding claims, wherein the administering comprises administering to the subject: (a) a first and second induction dose of the pharmaceutical composition, wherein the first induction dose is administered on Day 1, and (b) four or more maintenance doses of the pharmaceutical composition, wherein the first maintenance dose is administered about 1 week after the first induction dose, and wherein the second and subsequent maintenance doses are administered at about weekly intervals (QW) thereafter.

18. The method or pharmaceutical composition of claim 17, wherein the second induction dose is administered on about Day 4.

19. The method or pharmaceutical composition of any of the preceding claims, wherein the administering comprises administering to the subject: (a) a first, second, and third induction dose, wherein the first induction dose is administered on Day 1, and (b) four or more maintenance doses of the pharmaceutical composition, wherein the first maintenance dose is administered about 1 week after the first induction dose and the second and subsequent maintenance doses are administered at about weekly intervals (QW) thereafter.

20. The method or pharmaceutical composition of claim 19, wherein the second induction dose is administered on about Day 3 and the third induction dose is administered on about Day 5.

21. The method or pharmaceutical composition according to any of claims 11-20, wherein each dose administered comprises from about 2 mg to about 500 mg of the C5-binding protein.

22. The method or pharmaceutical composition according to any of claims 11-21, wherein each dose administered comprises from about 50 mg to about 450 mg of the C5-binding protein.

23. The method or pharmaceutical composition according to any of claims 11-22, wherein each dose administered comprises from about 100 mg to about 450 mg of the C5-binding protein.

24. The method or pharmaceutical composition according to any of claims 11-23, wherein each dose administered comprises from about 100 mg to about 350 mg of the C5-binding protein.

25. The method or pharmaceutical composition according to any of claims 14-24, wherein each induction dose comprises from about 100 mg to about 350 mg of the C5-binding protein.

26. The method or pharmaceutical composition according to any of claims 14-25, wherein each induction dose comprises about 100 mg, or about 150 mg, or about 300 mg of the C5-binding protein.

27. The method or pharmaceutical composition according to any of claims 14-26, wherein each maintenance dose comprises about 100 mg, or about 150 mg, or about 300 mg of the C5-binding protein.

28. The method or pharmaceutical composition of any of the preceding claims, wherein the C5-binding protein is administered to the subject at a dose and/or at a frequency effective to reduce the amount of free C5 in the subject.

29. The method or pharmaceutical composition of any of the preceding claims, wherein the C5-binding protein is administered to the subject at a dose and/or at a frequency effective to reduce the serum free C5 concentration to about 0.5 micrograms/mL or less.

30. The method or pharmaceutical composition of any of the preceding claims, wherein the C5-binding protein is administered to the subject at a dose and/or at a frequency effective to reduce the serum free C5 concentration to about 0.1 micrograms/mL or less.

31. The method or pharmaceutical composition of any of the preceding claims, wherein the C5-binding protein is administered to the subject at a dose and/or at a frequency effective to inhibit activation of the terminal complement pathway in the subject

32. The method or pharmaceutical composition of any of the preceding claims, wherein the C5-binding protein is administered to the subject at a dose and/or at a frequency effective to achieve a serum concentration of the C5-binding protein of from about 0.5 to about 5.0 micromolar.

33. The method or pharmaceutical composition of any of the preceding claims, wherein the C5-binding protein is administered to the subject at a dose and/or at a frequency effective to achieve a Cₘₐₓ of the C5-binding protein of from about 0.1 to about 10.0 micromolar.

34. The method or pharmaceutical composition of any of the preceding claims, wherein the C5-binding protein is administered to the subject at a dose and/or at a frequency effective to achieve a C_{trough} of the C5-binding protein of from about 0.1 to about 10 micromolar.

35. The method or pharmaceutical composition of any of the preceding claims, wherein the pharmaceutical composition comprises one or more additional components selected from the group consisting of: histidine, arginine, polysorbate 20, and water.

36. The method or pharmaceutical composition of any of the preceding claims, wherein the pharmaceutical composition comprises histidine, arginine, polysorbate 20, and water.

37. The method or pharmaceutical composition of any of the preceding claims, wherein the pharmaceutical composition comprises about 20 mM histidine, about 150 mM arginine, about 0.05% polysorbate 20, and water.

38. The method or pharmaceutical composition of any of the preceding claims, wherein the pharmaceutical composition comprises about 100 mg/ml of the C5-binding protein.

39. The method or pharmaceutical composition of any of the preceding claims, wherein the pH of the pharmaceutical composition is about 7.0.

40. The method or pharmaceutical composition of any of the preceding claims, wherein the pharmaceutical composition is stable when stored at about 2° C. to about 8° C.

41. The method or pharmaceutical composition of any of the preceding claims, wherein the pharmaceutical composition is stable for more than 6 months when stored at about 2° C. to about 8° C.

42. The method or pharmaceutical composition of any of the preceding claims, wherein the pharmaceutical composition is stable for more than 12 months when stored at about 2° C. to about 8° C.

43. The method or pharmaceutical composition of any of the preceding claims, wherein the pharmaceutical composition is stable for more than 18 months when stored at about 2° C. to about 8° C.

44. The method or pharmaceutical composition of any of the preceding claims, wherein the pharmaceutical composition is stable for up to about 36 hours when stored at about 15° C. to about 25° C.

45. The method or pharmaceutical composition of any of the preceding claims, wherein the pharmaceutical composition is stable for more than about 36 hours when stored at about 15° C. to about 25° C.


SEQUENCE LISTING (1 sequence)

SEQ ID NO: 1 - protein, 108 aa, synthetic construct
AEAKYAKEVLEAWDEIDRLPNLTIEQWLAFINKLDDDPSQSSELLSEAKKLSESQAPKVEGSLAEAKEAANAELDSYGVSDFYKRLIDKAKTVEGVEALKDAILAALP